(19) United States
(12) Patent Application Publication
(10) Pub. No.: US 2008/0227214 A1
(43) Pub. Date: Sep. 18, 2008

(54) Method for the Hydrocyanation of 1,3-Butadiene

(75) Inventors: Tim Jungkamp, Kapellen (BE); Robert Baumann, Mannheim (DE); Thorsten Schroder, Mannheim (DE); Michael Bartsch, Neustadt (DE); Gerd Haderlein, Grunstadt (DE); Hermann Luyken, Ludwigshafen (DE); Jens Scheidel, Hirschberg (DE)

Correspondence Address: CONNOLLY BOVE LODGE & HUTZ LLP, 1875 EYE STREET, N.W., SUITE 1100, WASHINGTON, DC, 20036, US

(73) Assignee: BASF AKTIENGESELLSCHAFT, LUDWIGSHAFEN (DE)

(21) Appl. No.: 10/586,473

(22) PCT Filed: Jan. 26, 2005
(86) PCT No.: PCT/EP05/00725

§ 371 Date: Jul. 18, 2006

(30) Foreign Application Priority Data
Jan. 29, 2004 (DE) 10-2004-004-672-7

(51) Int. Cl. G01N 31/16; C07C 253/10; G01J 5/02; G01N 9/00; G01N 30/02; G01N 25/18

(52) U.S. Cl. 436/109; 558/335; 558/338; 250/339.07; 73/32 R; 73/23.35; 374/44

(57) ABSTRACT

A process is described for preparing 3-pentenenitrile by hydrocyanating 1,3-butadiene in the presence of at least one catalyst, wherein unhydrocyanated 1,3-butadiene is removed from the effluent of the hydrocyanation and recycled into the process, and the recycled 1,3-butadiene is monitored for the content of hydrogen cyanide.

The present invention relates to a process for preparing 3-pentenenitrile by hydrocyanating 1,3-butadiene in the presence of at least one catalyst.

Adiponitrile, an important intermediate in nylon production, is prepared by double hydrocyanation of 1,3-butadiene. In a first hydrocyanation, 1,3-butadiene is reacted with hydrogen cyanide in the presence of nickel(0) which is stabilized with phosphorus ligands to give 3-pentenenitrile. In a second hydrocyanation, 3-pentenenitrile is subsequently reacted with hydrogen cyanide to give adiponitrile, likewise over a nickel catalyst, but with addition of a Lewis acid.

In the first hydrocyanation, 1,3-butadiene is used in a stoichiometric excess in relation to hydrogen cyanide in the hydrocyanation reaction. In the hydrocyanation, the hydrogen cyanide used is virtually fully depleted. However, a residual content of hydrogen cyanide of from 10 to 5000 ppm by weight remains in the reaction effluent from this hydrocyanation.

In the removal of the unconverted 1,3-butadiene, the undepleted hydrogen cyanide gets into the stream of the recycling of the 1,3-butadiene. This conveys an additional amount, unrecognized under some circumstances, of hydrogen cyanide back into the first hydrocyanation of 1,3-butadiene, so that increasingly higher contents of hydrogen cyanide can accumulate in the reaction mixture. In addition, problems can then occur in the recycling of the 1,3-butadiene with hydrogen cyanide present when the content of hydrogen cyanide in the 1,3-butadiene is too high, since unconverted hydrogen cyanide, when in a high content, can react with the nickel(0) catalyst used and then damage it irreversibly by formation of solids which comprise nickel(II) cyanide.

In addition, when unconverted hydrogen cyanide is present in a high content in the process for preparing 3-pentenenitrile, it can polymerize with a highly exothermic reaction and in some cases lead to vessel explosion.

Moreover, owing to its known toxicity, a high content of hydrogen cyanide in the stream of 1,3-butadiene leads to problems when the 1,3-butadiene is not to be recycled into the process and is withdrawn from the process.

It is accordingly an object of the present invention to provide a process for preparing 3-pentenenitrile by hydrocyanating 1,3-butadiene in the presence of at least one catalyst, which allows the above-described problems to be avoided and the process safety to be increased.

The achievement of this object starts from a process for preparing 3-pentenenitrile by hydrocyanating 1,3-butadiene in the presence of at least one catalyst. The process according to the invention comprises removing unhydrocyanated 1,3-butadiene from the effluent of the hydrocyanation and recycling it into the process, and determining the content of hydrogen cyanide in the recycled stream of 1,3-butadiene.

In the context of the present invention, a determination of the content of hydrogen cyanide in the recycled stream of 1,3-butadiene means that the content is measured preferably at regular intervals, more preferably permanently, and that, where a limiting value is exceeded, this exceedance is indicated and, if appropriate, suitable measures are initiated in order to prevent further contamination of the 1,3-butadiene with hydrogen cyanide. Such a limiting value is preferably 10% by weight, more preferably 7% by weight, in particular 5% by weight, of hydrogen cyanide, based in each case on the mixture of 1,3-butadiene and hydrogen cyanide. There may be a preliminary alarm even at values lower than those mentioned, for example 2.5% by weight or 1.5% by weight.

In the context of the present application, butadiene refers to 1,3-butadiene which comprises constituents which are also present in commercial 1,3-butadiene. In addition, pentenenitrile isomers may also be present. The content of pentenenitrile isomers is preferably less than 1% by weight, more preferably less than 0.5% by weight, in particular less than 1000 ppm by weight.

3-Pentenenitrile also refers to the corresponding isomers, for example 2-methyl-3-butenenitrile.

The process according to the invention is based on the discovery that hydrogen cyanide, on evaporation of 1,3-butadiene from the effluent of the hydrocyanation, gets into the vapor phase. It has been found in accordance with the invention that, even in the case of a fractional distillation of 1,3-butadiene from mixtures which comprise 1,3-butadiene (b.p.^{1013 mbar}=−4° C.) and hydrogen cyanide (b.p.^{1013 mbar}=+27° C.), despite a large boiling point difference of 31° C., hydrogen cyanide is always found in the top effluent. Hydrogen cyanide and 1,3-butadiene form a boiling point minimum azeotrope, so that, irrespective of the conditions under which the hydrocyanation effluent is partly evaporated, hydrogen cyanide always distills over in a mixture with 1,3-butadiene.

In industrial practice, it has been found to be difficult to detect hydrogen cyanide directly in the reaction effluent of the first hydrocyanation, since the presence of pentenenitriles, catalyst complexes, polymeric hydrogen cyanides and solids exceeds the capabilities of virtually all conceivable analytical methods. Especially the fouling in the presence of solids rules out a multitude of analytical methods which do not have a contactless measurement principle.

According to the invention, it has now been found that the content of hydrogen cyanide in the recycled stream of 1,3-butadiene is determined by at least one method which is selected from the group consisting of:
(1) near infrared transmission spectrometry in the liquid phase;
(2) middle infrared transmission spectrometry in the gas phase and/or liquid phase;
(3) ATR middle infrared spectrometry in the liquid phase;
(4) density measurement in the liquid phase, which is based on the difference in the densities of hydrogen cyanide and 1,3-butadiene;
(5) measurement of the thermal conductivity;
(6) measurement of the sound velocity;
(7) measurement of the dielectric permittivity;
(8) measurement of the refractive index;
(9) online gas chromatography determination;
(10) measurement of the heat capacity of the liquid phase;
(11) online sampling and Vollhardt or Liebig titration for hydrogen cyanide,
   and a sample of the recycled 1,3-butadiene is taken online.

In the context of the present invention, “taken” means that the content of hydrogen cyanide in the recycled 1,3-butadiene can also be determined in measurement systems which are flowed through or are contactless, as described in detail below.

In the context of this invention, online means that there is preferably no interruption of a stream in the process in order to take samples, since the suitable measurement probes are flowed through continuously or work contactlessly, or an automatic sampling system is used which, if appropriate, fills sampling vessels or analytical cuvettes at preferably regular intervals.

Particular preference is given to monitoring the content of hydrogen cyanide in the recycled 1,3-butadiene by measuring the dielectric permittivity using at least one apparatus for measuring the level by capacitive measurement methods.

For the process according to the invention, it is advantageous that the 1,3-butadiene to be monitored is substantially free of solids, since solids generally lead to blockages in online sampling systems and thus to reduced availability which is disadvantageous for plant safety, and prevent the use of optical methods because the particles, for example, prevent or greatly weaken the transmission of light. In this case, substantially free of solids means a solids content of at most 500 ppm by weight, more preferably at most 100 ppm by weight, in particular at most 10 ppm by weight.

The aforementioned methods for monitoring the content of hydrogen cyanide in the recycled 1,3-butadiene are more preferably carried out in product streams which are formed by evaporating a proportion of the reaction effluent, in which case the evaporated fraction can be condensed again and the analysis can take place in the resulting purified liquid phase.

This allows unintentional recycling of unrecognized amounts of hydrogen cyanide into the reactors for hydrocyanating 1,3-butadiene to be recognized and, as a consequence thereof, to be prevented by suitable measures. Such suitable measures are, for example, measures for increasing the hydrogen cyanide conversion in the hydrocyanation reaction, for example by increasing the temperature or by metering in additional, preferably fresh catalyst, or as shutdown of the hydrogen cyanide feed into the system, if appropriate with total shutdown of the hydrocyanation.

In a particular embodiment of the process according to the invention, the recycled stream of 1,3-butadiene is formed by evaporating at least a portion of the effluent of the hydrocyanation of 1,3-butadiene, in which case the evaporated proportion of the effluent of the hydrocyanation is, if appropriate, condensed again before the monitoring for hydrogen cyanide. A process which is particularly suitable for this purpose is described in DE-A-102 004 004 724. In addition, DE-A-102 004 004 718 describes a process for reducing the content of hydrogen cyanide in pentenenitrile-containing mixtures, wherein the reduction is effected by an azeotropic distillation of the hydrogen cyanide with 1,3-butadiene. The above-discussed azeotrope formation of hydrogen cyanide and 1,3-butadiene always results in hydrogen cyanide being present in the recycled 1,3-butadiene when hydrogen cyanide is present in the reaction effluent of the hydrocyanation. In a further preferred embodiment of the process according to the invention, at least a portion of the effluent of the hydrocyanation is therefore evaporated as an azeotrope of 1,3-butadiene and hydrogen cyanide.

From the measured concentration of hydrogen cyanide in the 1,3-butadiene which is recycled and the corresponding flow rates, it is possible to determine the concentration of hydrogen cyanide in the reactor itself and also the amount of hydrogen cyanide which is introduced with the recycled 1,3-butadiene in addition to the regular hydrogen cyanide feed into the reactors.

The hydrogen cyanide content is measured preferably in the gas phase of the condensate collecting vessel or in the liquid phase of the condensate collecting vessel or, in flooded operation, in the pumped circulation system of the condensate collecting vessel of the distillation apparatus for recovering the hydrogen cyanide-containing 1,3-butadiene from the effluent of the hydrocyanation.

The process according to the invention for hydrocyanating 1,3-butadiene is preferably carried out in the presence of at least one homogeneously dissolved nickel(0) complex having phosphorus ligands.

The Ni(0) complexes which contain phosphorus ligands and/or free phosphorus ligands are preferably homogeneously dissolved nickel(0) complexes.

The phosphorus ligands of the nickel(0) complexes and the free phosphorus ligands are preferably selected from mono- or bidentate phosphines, phosphites, phosphinites and phosphonites.

These phosphorus ligands preferably have the formula I:

P(X¹R¹)(X²R²)(X³R³) (I)

In the context of the present invention, compound I is a single compound or a mixture of different compounds of the aforementioned formula.

According to the invention, X¹, X², X³ each independently are oxygen or a single bond. When all of the X¹, X² and X³ groups are single bonds, compound I is a phosphine of the formula P(R¹R²R³) with the definitions of R¹, R² and R³ specified in this description.

When two of the X¹, X² and X³ groups are single bonds and one is oxygen, compound I is a phosphinite of the formula P(OR¹)(R²)(R³) or P(R¹)(OR²)(R³) or P(R¹)(R²)(OR³) with the definitions of R¹, R² and R³ specified hereinbelow.

When one of the X¹, X² and X³ groups is a single bond and two are oxygen, compound I is a phosphonite of the formula P(OR¹)(OR²)(R³) or P(R¹)(OR²)(OR³) or P(OR¹)(R²)(OR³) with the definitions of R¹, R² and R³ specified in this description.

In a preferred embodiment, all X¹, X² and X³ groups should be oxygen, so that compound I is advantageously a phosphite of the formula P(OR¹)(OR²)(OR³) with the definitions of R¹, R² and R³ specified hereinbelow.

According to the invention, R¹, R², R³ are each independently identical or different organic radicals. R¹, R² and R³ are each independently alkyl radicals preferably having from 1 to 10 carbon atoms, such as methyl, ethyl, n-propyl, i-propyl, n-butyl, i-butyl, s-butyl, t-butyl, aryl groups such as phenyl, o-tolyl, m-tolyl, p-tolyl, 1-naphthyl, 2-naphthyl, or hydrocarbyl, preferably having from 1 to 20 carbon atoms, such as 1,1′-biphenol, 1,1′-binaphthol. The R¹, R² and R³ groups may be bonded together directly, i.e. not solely via the central phosphorus atom. Preference is given to the R¹, R² and R³ groups not being bonded together directly.

In a preferred embodiment, R¹, R² and R³ are radicals selected from the group consisting of phenyl, o-tolyl, m-tolyl and p-tolyl. In a particularly preferred embodiment, a maximum of two of the R¹, R² and R³ groups should be phenyl groups.

In another preferred embodiment, a maximum of two of the R¹, R² and R³ groups should be o-tolyl groups.

Particularly preferred compounds I which may be used are those of the formula I a

(o-tolyl-O—)_{w}(m-tolyl-O—)ₓ(p-tolyl-O—)_{y}(phenyl-O—)_{z}P (I a)

where w, x, y and z are each a natural number and the following conditions apply: w+x+y+z=3 and w, z≦2.

Such compounds I a are, for example, (p-tolyl-O—)(phenyl-O—)₂P, (m-tolyl-O—)(phenyl-O—)₂P, (o-tolyl-O—)(phenyl-O—)₂P, (p-tolyl-O—)₂(phenyl-O—)P, (m-tolyl-O—)₂(phenyl-O—)P, (o-tolyl-O—)₂(phenyl-O—)P, (m-tolyl-O—)(p-tolyl-O—)(phenyl-O—)P, (o-tolyl-O—)(p-tolyl-O—)(phenyl-O—)P, (o-tolyl-O—)(m-tolyl-O—)(phenyl-O—)P, (p-tolyl-O—)₃P, (m-tolyl-O—)(p-tolyl-O—)₂P, (o-tolyl-O—)(p-tolyl-O—)₂P, (m-tolyl-O—)₂(p-tolyl-O—)P, (o-tolyl-O—)₂(p-tolyl-O—)P, (o-tolyl-O—)(m-tolyl-O—)(p-tolyl-O—)P, (m-tolyl-O—)₃P, (o-tolyl-O—)(m-tolyl-O—)₂P, (O-tolyl-O—)₂(m-tolyl-O—)P or mixtures of such compounds.

For example, mixtures comprising (m-tolyl-O—)₃P, (m-tolyl-O—)₂(p-tolyl-O—)P, (m-tolyl-O—)(p-tolyl-O—)₂P and (p-tolyl-O—)₃P may be obtained by reacting a mixture comprising m-cresol and p-cresol, in particular in a molar ratio of 2:1, as obtained in the distillative workup of crude oil, with a phosphorus trihalide, such as phosphorus trichloride.

In another, likewise preferred embodiment, the phosphorus ligands are the phosphites, described in detail in DE-A 199 53 058, of the formula I b:

P(O—R¹)ₓ(O—R²)_{y}(O—R³)_{z}(O—R⁴) (I b)

where
R¹: aromatic radical having a C₁-C₁₈-alkyl substituent in the o-position to the oxygen atom which joins the phosphorus atom to the aromatic system; or having an aromatic substituent in the o-position to the oxygen atom which joins the phosphorus atom to the aromatic system, or having a fused aromatic system in the o-position to the oxygen atom which joins the phosphorus atom to the aromatic system,
R²: aromatic radical having a C₁-C₁₈-alkyl substituent in the m-position to the oxygen atom which joins the phosphorus atom to the aromatic system, or having an aromatic substituent in the m-position to the oxygen atom which joins the phosphorus atom to the aromatic system, or having a fused aromatic system in the m-position to the oxygen atom which joins the phosphorus atom to the aromatic system, the aromatic radical bearing a hydrogen atom in the o-position to the oxygen atom which joins the phosphorus atom to the aromatic system,
R³: aromatic radical having a C₁-C₁₈-alkyl substituent in the p-position to the oxygen atom which joins the phosphorus atom to the aromatic system, or having an aromatic substituent in the p-position to the oxygen atom which joins the phosphorus atom to the aromatic system, the aromatic radical bearing a hydrogen atom in the o-position to the oxygen atom which joins the phosphorus atom to the aromatic system,
R⁴: aromatic radical which bears substituents other than those defined for R¹, R² and R³ in the o-, m- and p-position to the oxygen atom which joins the phosphorus atom to the aromatic system, the aromatic radical bearing a hydrogen atom in the o-position to the oxygen atom which joins the phosphorus atom to the aromatic system,
x: 1 or 2,
y, z, p: each independently 0, 1 or 2, with the proviso that x+y+z+p=3.

Preferred phosphites of the formula I b can be taken from DE-A 199 53 058. The R¹ radical may advantageously be o-tolyl, o-ethylphenyl, o-n-propylphenyl, o-isopropyl-phenyl, o-n-butylphenyl, o-sec-butylphenyl, o-tert-butylphenyl, (o-phenyl)phenyl or 1-naphthyl groups.

Preferred R² radicals are m-tolyl, m-ethylphenyl, m-n-propylphenyl, m-isopropylphenyl, m-n-butylphenyl, m-sec-butylphenyl, m-tert-butylphenyl, (m-phenyl)phenyl or 2-naphthyl groups.

Advantageous R³ radicals are p-tolyl, p-ethylphenyl, p-n-propylphenyl, p-isopropyl-phenyl, p-n-butylphenyl, p-sec-butylphenyl, p-tert-butylphenyl or (p-phenyl)phenyl groups.

The R⁴ radical is preferably phenyl. p is preferably zero. For the indices x, y, z and p in compound I b, there are the following possibilities:

| x | y | z | p |
|---|---|---|---|
| 1 | 0 | 0 | 2 |
| 1 | 0 | 1 | 1 |
| 1 | 1 | 0 | 1 |
| 2 | 0 | 0 | 1 |
| 1 | 0 | 2 | 0 |
| 1 | 1 | 1 | 0 |
| 1 | 2 | 0 | 0 |
| 2 | 0 | 1 | 0 |
| 2 | 1 | 0 | 0 |

Preferred phosphites of the formula I b are those in which p is zero, and R¹, R² and R³ are each independently selected from o-isopropylphenyl, m-tolyl and p-tolyl, and R⁴ is phenyl.

Particularly preferred phosphites of the formula I b are those in which R¹ is the o-isopropylphenyl radical, R² is the m-tolyl radical and R³ is the p-tolyl radical with the indices specified in the table above; also those in which R¹ is the o-tolyl radical, R² is the m-tolyl radical and R³ is the p-tolyl radical with the indices specified in the table; additionally those in which R¹ is the 1-naphthyl radical, R² is the m-tolyl radical and R³ is the p-tolyl radical with the indices specified in the table; also those in which R¹ is the o-tolyl radical, R² is the 2-naphthyl radical and R³ is the p-tolyl radical with the indices specified in the table; and finally those in which R¹ is the o-isopropylphenyl radical, R² is the 2-naphthyl radical and R³ is the p-tolyl radical with the indices specified in the table; and also mixtures of these phosphites.

Phosphites of the formula I b may be obtained by
a) reacting a phosphorus trihalide with an alcohol selected from the group consisting of R¹OH, R²OH, R³OH and R⁴OH or mixtures thereof to obtain a dihalophosphorous monoester,
b) reacting the dihalophosphorous monoester mentioned with an alcohol selected from the group consisting of R¹⁰H, R²OH, R³OH and R⁴OH or mixtures thereof to obtain a monohalophosphorous diester and
c) reacting the monohalophosphorous diester mentioned with an alcohol selected from the group consisting of R¹⁰H, R²OH, R³OH and R⁴OH or mixtures thereof to obtain a phosphite of the formula I b.

The reaction may be carried out in three separate steps. Equally, two of the three steps may be combined, i.e. a) with b) or b) with c). Alternatively, all of the steps a), b) and c) may be combined together.

Suitable parameters and amounts of the alcohols selected from the group consisting of R¹OH, R²OH, R³OH and R⁴OH or mixtures thereof may be determined readily by a few simple preliminary experiments.

Useful phosphorus trihalides are in principle all phosphorus trihalides, preferably those in which the halide used is Cl, Br, I, in particular Cl, and mixtures thereof. It is also possible to use mixtures of various identically or differently halogen-substituted phosphines as the phosphorus trihalide. Particular preference is given to PCl₃. Further details on the reaction conditions in the preparation of the phosphites I b and for the workup can be taken from DE-A 199 53 058.

The phosphites I b may also be used in the form of a mixture of different phosphites I b as a ligand. Such a mixture may be obtained, for example, in the preparation of the phosphites I b.

However, preference is given to the phosphorus ligand being multidentate, in particular bidentate. The ligand used therefore preferably has the formula II

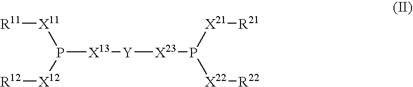

where
X¹¹, X¹², X¹³, X²¹, X²², X²³ are each independently oxygen or a single bond
R¹¹, R¹² are each independently identical or different, separate or bridged organic radicals
R²¹, R²² are each independently identical or different, separate or bridged organic radicals,
Y is a bridging group.

In the context of the present invention, compound II is a single compound or a mixture of different compounds of the aforementioned formula.

In a preferred embodiment, X¹¹, X¹², X¹³, X²¹, X²², X²³ may each be oxygen. In such a case, the bridging group Y is bonded to phosphite groups.

In another preferred embodiment, X¹¹ and X¹² may each be oxygen and X¹³ a single bond, or X¹¹ and X¹³ each oxygen and X¹² a single bond, so that the phosphorus atom surrounded by X¹¹, X¹² and X¹³ is the central atom of a phosphonite. In such a case, X²¹, X²² and X²³ may each be oxygen, or X²¹ and X²² may each be oxygen and X²³ a single bond, or X²¹ and X²³ may each be oxygen and X²² a single bond, or X²³ may be oxygen and X²¹ and X²² each a single bond, or X²¹ may be oxygen and X²² and X²³ each a single bond, or X²¹, X²² and X²³ may each be a single bond, so that the phosphorus atom surrounded by X²¹, X²² and X²³ may be the central atom of a phosphite, phosphonite, phosphinite or phosphine, preferably a phosphonite.

In another preferred embodiment, X¹³ may be oxygen and X¹¹ and X¹² each a single bond, or X¹¹ may be oxygen and X¹² and X¹³ each a single bond, so that the phosphorus atom surrounded by X¹¹, X¹² and X¹³ is the central atom of a phosphonite. In such a case, X²¹, X²² and X²³ may each be oxygen, or X²³ may be oxygen and X²¹ and X²² a single bond, or X²¹ may be oxygen and X²² and X²³ each a single bond, or X²¹, X²² and X²³ may each be a single bond, so that the phosphorus atom surrounded by X²¹, X²² and X²³ may be the central atom of a phosphite, phosphinite or phosphine, preferably a phosphinite.

In another preferred embodiment, X¹¹, X¹² and X¹³ may each be a single bond, so that the phosphorus atom surrounded by X¹¹, X¹² and X¹³ is the central atom of a phosphine. In such a case, X²¹, X²² and X²³ may each be oxygen, or X²¹, X²² and X²³ may each be a single bond, so that the phosphorus atom surrounded by X²¹, X²² and X²³ may be the central atom of a phosphite or phosphine, preferably a phosphine.

The bridging group Y is advantageously an aryl group which is substituted, for example by C₁-C₄-alkyl, halogen, such as fluorine, chlorine, bromine, halogenated alkyl, such as trifluoromethyl, aryl, such as phenyl, or is unsubstituted, preferably a group having from 6 to 20 carbon atoms in the aromatic system, in particular pyrocatechol, bis(phenol) or bis(naphthol).

The R¹¹ and R¹² radicals may each independently be identical or different organic radicals. Advantageous R¹¹ and R¹² radicals are aryl radicals, preferably those having from 6 to 10 carbon atoms, which may be unsubstituted or mono- or polysubstituted, in particular by C₁-C₄-alkyl, halogen, such as fluorine, chlorine, bromine, halogenated alkyl, such as trifluoromethyl, aryl, such as phenyl, or unsubstituted aryl groups.

The R²¹ and R²² radicals may each independently be identical or different organic radicals. Advantageous R²¹ and R²² radicals are aryl radicals, preferably those having from 6 to 10 carbon atoms, which may be unsubstituted or mono- or polysubstituted, in particular by C₁-C₄-alkyl, halogen, such as fluorine, chlorine, bromine, halogenated alkyl, such as trifluoromethyl, aryl, such as phenyl, or unsubstituted aryl groups.

The R¹¹ and R¹² radicals may each be separate or bridged. The R²¹ and R²² radicals may also each be separate or bridged. The R¹¹, R¹², R²¹ and R²² radicals may each be separate, two may be bridged and two separate, or all four may be bridged, in the manner described.

In a particularly preferred embodiment, useful compounds are those of the formula I, II, III, IV and V specified in U.S. Pat. No. 5,723,641. In a particularly preferred embodiment, useful compounds are those of the formula I, II, III, IV, V, VI and VII specified in U.S. Pat. No. 5,512,696, in particular the compounds used there in examples 1 to 31. In a particularly preferred embodiment, useful compounds are those of the formula I, II, III, IV, V, VI, VII, VIII, IX, X, XI, XII, XIII, XIV and XV specified in U.S. Pat. No. 5,821,378, in particular the compounds used there in examples 1 to 73.

In a particularly preferred embodiment, useful compounds are those of the formula I, II, III, IV, V and VI specified in U.S. Pat. No. 5,512,695, in particular the compounds used there in examples 1 to 6. In a particularly preferred embodiment, useful compounds are those of the formula I, II, III, IV, V, VI, VII, VIII, IX, X, XI, XII, XIII and XIV specified in U.S. Pat. No. 5,981,772, in particular the compounds used there in examples 1 to 66.

In a particularly preferred embodiment, useful compounds are those specified in U.S. Pat. No. 6,127,567 and the compounds used there in examples 1 to 29. In a particularly preferred embodiment, useful compounds are those of the formula I, II, III, IV, V, VI, VII, VIII, IX and X specified in U.S. Pat. No. 6,020,516, in particular the compounds used there in examples 1 to 33. In a particularly preferred embodiment, useful compounds are those specified in U.S. Pat. No. 5,959,135, and the compounds used there in examples 1 to 13.

In a particularly preferred embodiment, useful compounds are those of the formula I, II and III specified in U.S. Pat. No. 5,847,191. In a particularly preferred embodiment, useful compounds are those specified in U.S. Pat. No. 5,523,453, in particular the compounds illustrated there in formula 1, 2, 3, 4, 5, 6, 7, 8, 9, 10, 11, 12, 13, 14, 15, 16, 17, 18, 19, 20 and 21. In a particularly preferred embodiment, useful compounds are those specified in WO 01/14392, preferably the compounds illustrated there in formula V, VI, VII, VIII, IX, X, XI, XII, XIII, XIV, XV, XVI, XVII, XXI, XXII, XXIII.

In a particularly preferred embodiment, useful compounds are those specified in WO 98/27054. In a particularly preferred embodiment, useful compounds are those specified in WO 99/13983. In a particularly preferred embodiment, useful compounds are those specified in WO 99/64155.

In a particularly preferred embodiment, useful compounds are those specified in the German patent application DE 100 380 37. In a particularly preferred embodiment, useful compounds are those specified in the German patent application DE 100 460 25. In a particularly preferred embodiment, useful compounds are those specified in the German patent application DE 101 502 85.

In a particularly preferred embodiment, useful compounds are those specified in the German patent application DE 101 502 86. In a particularly preferred embodiment, useful compounds are those specified in the German patent application DE 102 071 65. In a further particularly preferred embodiment of the present invention, useful phosphorus chelate ligands are those specified in US 2003/0100442 A1.

In a further particularly preferred embodiment of the present invention, useful phosphorus chelate ligands are those specified in the German patent application reference no. DE 103 50 999.2 of Oct. 30, 2003, which has an earlier priority date but had not been published at the priority date of the present invention.

The compounds I, I a, I b and II described and their preparation are known per se. Phosphorus ligands used may also be mixtures comprising at least two of the compounds I, I a, I b and II.

In a particularly preferred embodiment of the process according to the invention, the phosphorus ligand of the nickel(0) complex and/or the free phosphorus ligand is selected from tritolyl phosphite, bidentate phosphorus chelate ligands and the phosphites of the formula I b

P(O—R¹)ₓ(O—R²)_{y}(O—R³)_{z}(O—R⁴)ₚ (I b)

where R¹, R² and R³ are each independently selected from o-isopropylphenyl, m-tolyl and p-tolyl, R⁴ is phenyl; x is 1 or 2, and y, z, p are each independently 0, 1 or 2 with the proviso that x+y+z+p=3; and mixtures thereof.

The hydrocyanation may be carried out in any suitable apparatus known to those skilled in the art. Useful apparatus for the reaction is customary apparatus, as described, for example, in: Kirk-Othmer, Encyclopedia of Chemical Technology, 4th Ed., Vol. 20, John Wiley & Sons, New York 1996, pages 1040 to 1055, such as stirred tank reactors, loop reactors, gas circulation reactors, bubble column reactors or tubular reactors, in each case if appropriate with apparatus to remove heat of reaction. The reaction may be carried out in a plurality of, such as two or three, apparatuses.

In a preferred embodiment of the process according to the invention, advantageous reactors have been found to be those having backmixing characteristics or batteries of reactors having backmixing characteristics. It has been found that particularly advantageous batteries of reactors having backmixing characteristics are those which are operated in crossflow mode in relation to the metering of hydrogen cyanide.

The hydrocyanation may be carried out in batch mode, continuously or in semibatchwise operation.

Preference is given to carrying out the hydrocyanation continuously in one or more stirred process steps. When a plurality of process steps is used, it is preferred that the process steps are connected in series. In this case, the product from one process step is transferred directly into the next process step. The hydrogen cyanide may be added directly into the first process step or between the individual process steps.

When the hydrocyanation is carried out in semibatchwise operation, it is preferred that the reactor is initially charged with the catalyst components and 1,3-butadiene, while hydrogen cyanide is metered into the reaction mixture over the reaction time.

The hydrocyanation may be carried out in the presence or in the absence of a solvent. When a solvent is used, the solvent should be liquid and inert toward the unsaturated compounds and the at least one catalyst at the given reaction temperature and the given reaction pressure. In general, the solvents used are hydrocarbons, for example benzene or xylene, or nitrites, for example acetonitrile or benzonitrile. However, preference is given to using a ligand as the solvent.

The hydrocyanation may be carried out by charging the apparatus with all reactants. However, it is preferred when the apparatus is filled with the at least one catalyst, 1,3-butadiene and, if appropriate, the solvent. The gaseous hydrogen cyanide preferably floats over the surface of the reaction mixture or is preferably passed through the reaction mixture. A further procedure for charging the apparatus is the filling of the apparatus with the at least one catalyst, hydrogen cyanide and, if appropriate, the solvent, and slowly feeding the 1,3-butadiene to the reaction mixture. Alternatively, it is also possible that the reactants are introduced into the reactor and the reaction mixture is brought to the reaction temperature at which the hydrogen cyanide is added to the mixture in liquid form. In addition, the hydrogen cyanide may also be added before heating to reaction temperature. The reaction is carried out under conventional hydrocyanation conditions for temperature, atmosphere, reaction time, etc.

The hydrocyanation is carried out preferably at pressures of from 0.1 to 500 MPa, more preferably from 0.5 to 50 MPa, in particular from 1 to 5 MPa. The reaction is carried out preferably at temperatures of from 273 to 473 K, more preferably from 313 to 423 K, in particular at from 333 to 393 K. It has been found that advantageous average mean residence times of the liquid reactor phase are in the range from 0.001 to 100 hours, preferably from 0.05 to 20 hours, more preferably from 0.1 to 5 hours, in each case per reactor.

In one embodiment, the hydrocyanation may be performed in the liquid phase in the presence of a gas phase and, if appropriate, of a solid suspended phase. The starting materials, hydrogen cyanide and 1,3-butadiene, may in each case be metered in liquid or gaseous form.

In a further embodiment, the hydrocyanation may be carried out in the liquid phase, in which case the pressure in the reactor is such that all reactants such as 1,3-butadiene, hydrogen cyanide and the at least one catalyst are metered in liquid form and are present in the liquid phase in the reaction mixture. A solid suspended phase may be present in the reaction mixture and may also be metered together with the at least one catalyst, for example consisting of degradation products of the catalyst system, comprising nickel(II) compounds inter alia.

The present invention further relates to the use of at least one method which is selected from the group consisting of:
(1) near infrared transmission spectrometry in the liquid phase;
(2) middle infrared transmission spectrometry in the gas phase and/or liquid phase;
(3) ATR middle infrared spectrometry in the liquid phase;
(4) density measurement in the liquid phase, which is based on the difference in the densities of hydrogen cyanide and 1,3-butadiene;
(5) measurement of the thermal conductivity;
(6) measurement of the sound velocity;
(7) measurement of the dielectric permittivity;
(8) measurement of the refractive index;
(9) online gas chromatography determination;
(10) measurement of the heat capacity of the liquid phase;
(11) online sampling and Vollhardt or Liebig titration for hydrogen cyanide,
   for monitoring the content of hydrogen cyanide in streams which comprise 1,3-butadiene and hydrogen cyanide.

Measurement processes by the methods 1, in some cases 2, 3 to 5 and 8 to 11, are preferably effected by flow through a suitable sampling system which meters samples to the particular instruments, for example at preferably regular intervals. These processes may also be effected by direct flow through a suitable measurement apparatus, so that no sampling system is needed.

The measurement methods 6 and 7 are preferably performed using measuring probes which are not in contact with the product and thus preferably disposed outside apparatus which is flowed through. These measurements are preferably effected on an apparatus or a pipeline, more preferably a pipeline in the pumped circulation system of the condensate collecting vessel at the top of the distillation apparatus for recovering the hydrogen cyanide-containing 1,3-butadiene, or a reservoir vessel for hydrogen cyanide-containing 1,3-butadiene. The measurement point is preferably free of gas phase, and is more preferably a pipeline in flooded operation.

The calibration of the measurement probes is effected by introducing preferably a plurality of test mixtures of known content and/or known measurement parameters, such as sound velocity or dielectric permittivity, successively into the measurement point while recording calibration curves.

Particular preference is given to effecting the monitoring of the content of hydrogen cyanide in the recycled 1,3-butadiene by measuring the dielectric permittivity with an apparatus for measuring the level by capacitive measurement processes.

Suitable measurement probes for determining the dielectric permittivity are, for example, level measurement probes of the Endress+Hauser Multicap DC16 or Vega EL21 brands.

To calibrate the suitable measurement probes, suitable test mixtures are used.

These aforementioned measurement methods are preferably used in a process for preparing 3-pentenenitrile by hydrocyanating 1,3-butadiene in the presence of at least one catalyst. This measurement of hydrogen cyanide in streams which are substantially free of solids are preferred.

In a preferred embodiment of the process according to the invention, the content of hydrogen cyanide is measured in the stream which comprises 1,3-butadiene and is recycled into the hydrocyanation by measuring the relative dielectric permittivity using an instrument for measuring the level by capacitive measurement processes.

### EXAMPLE 1

### Measurement of Hydrogen Cyanide in Butadiene

The example describes measurements with probes for capacitive level measurement from two manufacturers (from Endress+Hauser, model: Multicap DC16; from Vega, model: EL21). The probes were installed in alternation in a thermostatable DN50 tube which was charged with mixtures of butadiene and hydrogen cyanide. The signals of the probes in the 0 to 10° C. temperature range and 0 to 5% by weight hydrogen cyanide concentration range were determined. For the measurements, unstabilized, distilled hydrogen cyanide and butadiene dried and destabilized over F200 alumina from Almatis according to the examples of DE-A-102 004 004 684 were used.

Butadiene was conducted in a circulation stream within the apparatus. In this circulation system was disposed a vessel B1 (designed as a DN50×500 mm jacketed tube) which was equipped with the particular capacitive level probe and also a thermometer and a manometer. The vessel B1 was cooled via the jacket with a cryostat. Butadiene was withdrawn from the vessel B1 via a gear pump P1 (working range from 0.5 to 5 l/min) and conveyed back into the vessel B1 via a liquid-gas sampler fitting. The pump P1 was equipped with an overflow valve Y1 (pₑ=4 bar) with recycling to the suction side of the pump. The pump head was cooled to approx. −5° C. by trace cooling. B1 was in flooded operation. The air reservoir used was a sightglass in the venting line. B1 was depressurized into the offgas line with an overflow valve Y2 (pₑ=2 bar). B1 was protected by a safety valve Y3 at pₑ=5 bar.

For the measurements with the particular probes, hydrogen cyanide was metered into the butadiene circuit. This metering was effected via a liquid-gas bomb B2 (V=25 ml). The bomb had been charged beforehand with a stock solution of small amounts of hydrogen cyanide in butadiene. Adjustment of the sampling path brought the material from B2 into the circulation system around B1. After the metered addition, the amount of hydrogen cyanide added was mixed in by the pumped circulation.

The measured signals of the probes were converted to an analog signal. The extent to which the measurement range of the probes was utilized was specified hereinbelow. The measurement range was calibrated beforehand by measurement in the empty system and in the circulation system filled exclusively with chloroform.

| | Hydrogen cyanide | | |
|---|---|---|---|
| | concentration in | | |
| | butadiene | Temperature | Output signal |
| Measurement probe | [% by weight] | [° C.] | [%] |
| Endress & Hauser | 0.0% | 0° C. | 26.2% |
| Multicap DC16 | | | |
| Endress & Hauser | 0.0% | 5° C. | 25.9% |
| Multicap DC16 | | | |
| Endress & Hauser | 0.0% | 10° C. | 25.6% |
| Multicap DC16 | | | |
| Endress & Hauser | 0.0% | 15° C. | 25.3% |
| Multicap DC16 | | | |
| Endress & Hauser | 0.8% | 10° C. | 30.5% |
| Multicap DC16 | | | |
| Endress & Hauser | 1.5% | 1° C. | 36.5% |
| Multicap DC16 | | | |
| Endress & Hauser | 1.5% | 5° C. | 35.8% |
| Multicap DC16 | | | |
| Endress & Hauser | 1.5% | 10° C. | 34.8% |
| Multicap DC16 | | | |
| Endress & Hauser | 3.2% | −1° C. | 50.1% |
| Multicap DC16 | | | |
| Endress & Hauser | 3.2% | 5° C. | 48.9% |
| Multicap DC16 | | | |
| Endress & Hauser | 3.2% | 10° C. | 47.3% |
| Multicap DC16 | | | |
| Endress & Hauser | 5.1% | 0° C. | 74.0% |
| Multicap DC16 | | | |
| Endress & Hauser | 5.1% | 5° C. | 71.0% |
| Multicap DC16 | | | |
| Endress & Hauser | 5.1% | 10° C. | 68.0% |
| Multicap DC16 | | | |
| Vega EL21 | 0.0% | 5° C. | 27.9% |
| Vega EL21 | 2.1% | 5° C. | 42.1% |
| Vega EL21 | 4.2% | 5° C. | 61.5% |
| Vega EL21 | 6.6% | 5° C. | 87.8% |

### EXAMPLE 2

### Liquid Phase IR

In a conventional FT-IR instrument, a pressure cuvette (p_{e,max}=25 bar) with cadmium selenide windows and 0.1 mm cuvette length was installed. After evacuation of the measurement cell, a 5 ml sample was injected via a sampler. In addition, it was also possible to introduce a sample from a syringe via a septum. The absorption was measured.

Spectra for hydrogen cyanide calibration with the cuvette filled with 3PN were recorded as the background. The concentration of hydrogen cyanide was measured by integration of the absorption band in the 2070 to 2110 cm⁻¹ range. Below 500 ppm by weight, the background noise was too high to be able to detect the hydrogen cyanide band.

Increase experiments with hydrogen cyanide in butadiene give the following measurements:

| Hydrogen cyanide concentration in | |
|---|---|
| butadiene | Absorption |
| 500 ppm by weight | 0.028 |
| 1200 ppm by weight | 0.042 |
| 3700 ppm by weight | 0.11 |
| 1% by weight | 0.32 |
| 5% by weight | 1.2 |
| 10% by weight | 2.0 |

After this calibration, a 5 ml sample from the circulation system described in example 1 was flushed into the empty bomb, which had also been used to introduce the stock solution of hydrogen cyanide in butadiene into the circulation system. From this bomb, application of nitrogen with a pressure of 5 bar compressed a sufficient sample volume into the cuvette. The circulation system from which the sample was taken had 1.5% by weight hydrogen cyanide at the time of sampling (calculation from metered amount of stock solution). The absorption determined in the IR instrument was 0.51.

1. A process for preparing 3-pentenenitrile by hydrocyanating 1,3-butadiene in the presence of at least one catalyst, the process comprising: removing unhydrocyanated 1,3-butadiene from the effluent of a hydrocyanation process and recycling the 1,3-butadiene to the hydrocyanation process by evaporating at least a portion of the effluent of the hydrocyanation, and at least a portion of the effluent of the hydrocyanation being evaporated as an azeotrope of 1,3-butadiene and hydrogen cyanide, the content of hydrogen cyanide being determined in the recycled stream of 1,3-butadiene.

2. The process according to claim 1, wherein the content of hydrogen cyanide in the recycled stream of 1,3-butadiene is determined by at least one method selected from the group consisting of:
near infrared transmission spectrometry in the liquid phase;
middle infrared transmission spectrometry in the gas phase and/or liquid phase;
ATR middle infrared spectrometry in the liquid phase;
density measurement in the liquid phase, which is based on the difference in the densities of hydrogen cyanide and 1,3-butadiene;
measurement of the thermal conductivity;
measurement of the sound velocity;
measurement of the dielectric permittivity;
measurement of the refractive index;
online gas chromatography determination;
measurement of the heat capacity of the liquid phase;
online sampling and Vollhardt or Liebig titration for hydrogen cyanide, and a sample of the recycled 1,3-butadiene is obtained online.

3. The process according to claim 1, wherein the monitoring of the content of hydrogen cyanide in the recycled stream of 1,3-butadiene is determined by measuring the relative dielectric permittivity using at least one apparatus for measuring the level by capacitative measurement processes.

4. The process according to claim 1, wherein the 1,3-butadiene to be monitored is substantially free of solids.

5. The process according to claim 1, wherein the hydrocyanation is conducted in the presence of at least one homogeneously dissolved nickel(0) complex having phosphorus ligands.

6. The process according to claim 5, wherein the phosphorus ligands are selected from the group consisting of mono- or bidentate phosphites, phosphonites, phosphines or phosphinites.

7. The use of at least one analytical method which is selected from the group consisting of:
near infrared transmission spectrometry in the liquid phase;
middle infrared transmission spectrometry in the gas phase and/or liquid phase;
ATR middle infrared spectrometry in the liquid phase;
density measurement in the liquid phase, which is based on the difference in the densities of hydrogen cyanide and 1,3-butadiene;
measurement of the thermal conductivity;
measurement of the sound velocity;
measurement of the dielectric permittivity;
measurement of the refractive index;
online gas chromatography determination;
measurement of the heat capacity of the liquid phase;
online sampling and Liebig titration for hydrogen cyanide,
for monitoring the content of hydrogen cyanide in streams which comprise 1,3-butadiene and hydrogen cyanide.

8. The use according to claim 7, wherein the monitoring is conducted in a process for preparing 3-pentenenitrile by hydrocyanating 1,3-butadiene in the presence of at least one catalyst.

9. The process according to claim 1, wherein the evaporated portion of the effluent of the hydrocyanation is condensed before the monitoring for hydrogen cyanide.

10. The process according to claim 3, wherein the 1,3-butadiene to be monitored is substantially free of solids.
